(19) United States
(12) Patent Application Publication
(10) Pub. No.: US 2013/0143288 A1
(43) Pub. Date: Jun. 6, 2013

(54) ISOLATED BACTERIA, METHODS FOR USE, AND METHODS FOR ISOLATION

(71) Applicants: David A. Mullin, New Orleans, LA (US); Harshad R. Velankar, Whitefield (IN)

(72) Inventors: David A. Mullin, New Orleans, LA (US); Harshad R. Velankar, Whitefield (IN)

(73) Assignee: The Administrators of the Tulane Educational Fund, New Orleans, LA (US)

(21) Appl. No.: 13/816,231

(22) PCT Filed: Aug. 11, 2011
(86) PCT No.: PCT/US11/47372

§ 371 Date: Feb. 24, 2013

Related U.S. Application Data
(60) Provisional application No. 61/401,395, filed on Aug. 12, 2010.

(51) Int. Cl. C12N 1/20

(52) U.S. Cl. CPC C12N 1/20
USPC 435/140; 435/252.7; 435/160; 435/141

(57) ABSTRACT

Isolated bacteria are disclosed. The isolated bacteria are aerotolerant and can produced butanol. Methods are disclosed to produce generated chemicals. Methods are disclosed to isolate aerotolerant bacteria.

### CROSS-REFERENCE TO RELATED APPLICATIONS

This application claims the benefit of U.S. Provisional Application No. 61/401,395, filed Aug. 12, 2010, which is herein incorporated by reference in its entirety.

### BACKGROUND

Butanol can be a useful chemical. For example, it can be used as a direct substitute for gasoline in gasoline burning vehicles. Also, butanol can be transported in existing fuel pipelines because it can be minimally corrosive and relatively insoluble in water.

Some bacteria used to produce butanol in the biomass conversion process belong to the genus Clostridium, which includes some anaerobic strains (e.g., Clostridium acetobutylicum). Current methods of acetone-butanol-ethanol (ABE) fermentation can occur under strict anaerobic conditions, and thus may raise the costs associated with butanol production. Like ethanol production, current methods of producing butanol can require biomass sources that compete with the human food supply.

Some embodiments of the invention may, but are not required to, address one or more of the above.

### SUMMARY

Some embodiments of the invention include isolated bacteria of the Clostridium genus, where the isolated bacteria (1) are aerotolerant and (2) can produce one or more generated chemicals; wherein one of the one or more generated chemicals is butanol. In some instances, the isolated bacteria are the bacterial strain TU-103 (ATCC ______). The 16S rRNA gene of the isolated bacteria can sometimes comprises SEQ ID NO:1.

In certain embodiments, the isolated bacteria can produce additional generated chemicals selected from the group consisting of acetone, butyric acid, and acetic acid. In some instances, the isolated bacteria can produce butanol at a concentration of at least about 3.0 gl⁻¹. In other embodiments, the isolated bacteria can produce acetone at a concentration of at least about 0.1 gl⁻¹. In some instances, the isolated bacteria can survive in a growth media comprising a percent of dissolved oxygen (DO) that is at least 0.1%. In other instances, the isolated bacteria can grow in a growth media comprising a percent of DO that is at least 0.1%. In still other embodiments, the isolated bacteria can survive exposure to air for at least about 1 minute. In certain embodiments, the isolated bacteria can hydrolyze one or more cellulose substrates or metabolize starch.

Some embodiments of the present invention include compositions comprising isolated bacteria, which in some instances are substantially pure.

Other embodiments of the present invention include methods for producing one or more generated chemicals comprising growing the isolated bacteria (that is aerotolerant and that can produce one or more generated chemicals) in a growth media, and recovering at least one of the one or more generated chemicals. In some of the embodiments, one or more generated chemicals comprise one or more selected from the group consisting of butanol, acetone, butyric acid, and acetic acid. In other embodiments, the DO in the growth media is at least 0.1%. In some instances, DO is removed from the growth media by the isolated bacteria. Growing the isolated bacteria can sometimes occur under anaerobic growth conditions. In some instances, butanol is produced in amount of at least 3.0 gl⁻¹. In still other embodiments, acetone is produced in an amount of at least 0.1 gl⁻¹. Sometimes, the isolated bacteria hydrolyze one or more cellulose substrates or metabolize starch.

Other embodiments of the invention include methods for isolating aerotolerant bacteria from a sample comprising exposing a sample to an isolating atmosphere that comprises at least trace amounts of O₂. In some embodiments of this method, the isolating atmosphere is in a glove box. In some instances, the method further comprises exposing the sample to heat, ethanol, or both.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1 shows the morphological features of a vegetative cell at 24 hours during cultivation of strain TU-103 in semisynthetic broth (P2Y) containing 20 gl⁻¹ glucose at 32° C.

FIG. 2 shows the morphological features of a cigar-shaped rod cell at 48 hours during cultivation of strain TU-103 in semisynthetic broth (P2Y) containing 20 gl⁻¹ glucose at 32° C.

FIG. 3 shows the morphological features of sporulation in rods from 48 hours to 120 hours during cultivation of strain TU-103 in semisynthetic broth (P2Y) containing 20 gl⁻¹ glucose at 32° C.

FIG. 4 shows a cladeogram in the form of an unrooted tree showing phylogenetic relationships of strain TU-103 to other clostridial strains based on 16S rRNA sequence comparisons.

FIG. 5 shows determination of ▪ glucose (gl⁻¹), □ dry cell mass (gl⁻¹), butanol (gl⁻¹), ◯ acetone (gl⁻¹) during fermentation by strain TU-103 at 32° C. in P2Y broth containing glucose at 20 gl⁻¹.

FIG. 6 shows determination ▪ glucose (gl⁻¹), □ dry cell mass (gl⁻¹), butanol (gl⁻¹), ◯ acetone (gl⁻¹) during fermentation by strain TU-103 at 32° C. in P2Y broth containing glucose at 30 gl⁻¹.

FIG. 7 shows determination of ▪ glucose (gl⁻¹), □ dry cell mass (gl⁻¹), butanol (gl⁻¹), ◯ acetone (gl⁻¹) during fermentation by strain TU-103 at 32° C. in P2Y broth containing glucose at 40 gl⁻¹.

FIG. 8 shows determination ▪ glucose (gl⁻¹), □ dry cell mass (gl⁻¹), butanol (gl⁻¹), ◯ acetone (gl⁻¹) during fermentation by strain TU-103 at 32° C. in P2Y broth containing glucose at 50 gl⁻¹.

FIG. 9 shows the effect of 89% air headspace volume on TU-103 cell growth and butanol and acetone production. □ biomass (gl⁻¹), butanol (gl⁻¹), ◯ acetone (gl⁻¹), and [symbol] DO concentrations at 89% air headspace volume incubated at 32° C., for 120 hours.

FIG. 10 shows the effect of 43% air headspace volume on TU-103 cell growth and butanol and acetone production. □ biomass (gl⁻¹), butanol (gl⁻¹), ◯ acetone (gl⁻¹), and [symbol] DO concentrations at 43% air headspace volume incubated at 32° C., for 120 hours.

FIG. 11 shows the effect of 0% air headspace volume on TU-103 cell growth and butanol and acetone production. □ biomass (gl⁻¹), butanol (gl⁻¹), ◯ acetone (gl⁻¹), and [symbol] DO concentrations at 0% air headspace volume (i.e., anaerobic condition) incubated at 32° C., for 120 hours.

FIG. 12 shows bioreactor fermentations of (A) Strain TU-103 and (B) Clostridium beijerinckii NCIMB 8052, and determination of butanol (closed circles), acetone (open circles), acetic acid (open diamond), butyric acid (closed diamond), biomass (open squares), ethanol (closed triangles) concentrations (gl⁻¹) and pH (closed squares) under anaerobiosis (0.05 vvm N₂) in P2Y at 32° C., 200 rpm for 80 hours.

FIG. 13 shows bioreactor fermentations of Strain TU-103 in P2Y with (A) 5% and (B) 21% (v/v) dissolved oxygen (open triangles) levels and the determination of butanol (closed circles), acetone (open circles), acetic acid (open diamonds), butyric acid (closed diamonds), biomass (open squares) concentrations (gl⁻¹) and pH (closed squares) under anaerobiosis (0.05 vvm N₂) at 32° C., 200 rpm for 80 hours.

### DETAILED DESCRIPTION

Some embodiments of the invention include isolated bacteria that are aerotolerant (i.e., the bacteria are not strict anaerobes and are able to survive and grow in the presence of O₂ including dissolved oxygen) and can produce one or more generated chemicals. As used herein, “dissolved oxygen” means O₂ dissolved in a liquid, gel, film, or solid, including but not limited to growth media. Growth media can be of any suitable form, including but not limited to liquid, gel, film, or solid. In some embodiments, the isolated bacteria are of the Clostridium genus. In certain embodiments, the isolated bacteria are the bacterial strain TU-103 (ATCC ______). In some embodiments, the generated chemical can include but is not limited to butanol, acetone, butyric acid, ethanol, acetic acid, ethanol, lactic acid, lactose, organic acids, alcohols, carbon dioxide, hydrogen, isopropanol, degradation products of cellulose or starch, or hydrolytic cleavage products of cellulose or starch. In some embodiments the isolated bacteria can produce one or more of the generated chemicals in any suitable amount, including but not limited to about 3.0 gl⁻¹, about 4.0 gl⁻¹, about 5.0 gl⁻¹, about 6.0 gl⁻¹, about 7.0 gl⁻¹, about 8.0 gl⁻¹, about 9.0 gl⁻¹, about 10.0 gl⁻¹, about 11.0 gl⁻¹, about 12.0 gl⁻¹, about 12.5 gl⁻¹, about 13.0 gl⁻¹, about 13.5 gl⁻¹, about 14.0 gl⁻¹, about 14.5 gl⁻¹, about 15.0 gl⁻¹, about 20.0 gl⁻¹, about 25.0 gl⁻¹, about 30.0 gl⁻¹, about 35.0 gl⁻¹, or about 40.0 gl⁻¹. In some embodiments, the isolated can produce butanol in any suitable amount, including but not limited to at least about 3.0 gl⁻¹, at least about 5.0 gl⁻¹, at least about 10.0 gl⁻¹, about 3.0 gl⁻¹, about 4.0 gl⁻¹, about 5.0 gl⁻¹, about 6.0 gl⁻¹, about 7.0 gl⁻¹, about 8.0 gl⁻¹, about 9.0 gl⁻¹, about 10.0 gl⁻¹, about 11.0 gl⁻¹, about 12.0 gl⁻¹, about 12.5 gl⁻¹, about 13.0 gl⁻¹, about 13.5 gl⁻¹, about 14.0 gl⁻¹, about 14.5 gl⁻¹, about 15.0 gl⁻¹, about 20.0 gl⁻¹, about 25.0 gl⁻¹, about 30.0 gl⁻¹, about 35.0 gl⁻¹, or about 40.0 gl⁻¹. In some embodiments, the isolated bacetria can produce acetone in any suitable amount, including but not limited to at least about 0.1 gl⁻¹, at least about 1.0 gl⁻¹, at least about 2.0 gl⁻¹, about 0.1 gl⁻¹, about 0.5 gl⁻¹, about 1.0 gl⁻¹, about 1.5 gl⁻¹, about 2.0 gl⁻¹, about 2.2 gl⁻¹, about 2.5 gl⁻¹, about 3.0 gl⁻¹, about 3.5 gl⁻¹, about 4.0 gl⁻¹, about 4.5 gl⁻¹, or about 5.0 gl⁻¹.

In some embodiments, the isolated bacteria can grow under aerobic conditions or under anaerobic conditions. In other embodiments, the isolated bacteria can produce one or more generated chemicals under aerobic conditions or under anaerobic conditions. In some embodiments, the isolated bacteria can grow in the presence of a suitable amount of DO. In other embodiments, the isolated bacteria can produce one or more generated chemicals in the presence of a suitable amount of DO. In some instances, the isolated bacteria can grow in growth media with a percent dissolved oxygen (DO) (v/v) of at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%. In some instances, the isolated bacteria can produce one or more generated chemicals in chemical media with a percent dissolved oxygen (DO) of at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%.

Other embodiments of the isolated bacteria can include the ability to remove dissolved oxygen from growth media, which in some instances may result in anaerobic conditions. Dissolved oxygen can be removed by the isolated bacteria by any suitable mechanism, including but not limited to using superoxide dismutase (e.g., with any suitable activity including but not limited to about 0.05, about 0.10, about 0.12, about 0.15, about 0.20, about 0.30, or about 0.50 U per milligram protein) or using catalase. In some instances, the catalase activity is not dependent on the presence of hemin. Still other embodiments include the ability of the isolated bacteria to survive exposure to air for a suitable period of time including but not limited to, at least about 1 minute, at least about 1 hour, about 1 minute, about 30 minutes, about 1 hour, about 12 hours, about 24 hours, about 48 hours, about 72 hours, about 96 hours, about 120 hours, about 240 hours, or about 480 hours.

Other embodiments of the inventions provide isolated bacteria that can hydrolyze one or more cellulose substrates, including but not limited to any suitable cellulose substrate, such as microcrystalline cellulose (C20), carboxymethyl cellulose (CMC), colloidal cellulose (CCOL), phosphoric acid treated cellulose (PASC), cotton fibers (CF), or filter paper (FP). Still other embodiments include isolated bacteria that can metabolize starch. Still other embodiments include isolated bacteria that can hydrolyze one or more cellulose substrate, metabolize starch, or both, and produce one or more generated chemicals (such as but not limited to butanol, acetone, or both).

In other embodiments of the invention, the isolated bacteria can produce acetone and butanol in the presence of DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%). In other embodiments of the invention, the isolated bacteria can grow in the presence of DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%). In still other embodiments of the invention, the isolated bacteria can grow and can produce acetone and butanol in the presence of DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%). In other embodiments of the invention, the isolated bacteria can establish anoxic conditions after initial cultivation in DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%). In other embodiments of the invention, the isolated bacteria can establish anoxic conditions after initial cultivation in DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%) and produce acetone and butanol in the anoxic condition. In other embodiments of the invention, the isolated bacteria can establish anoxic conditions after initial cultivation in DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%) and produce acetone and butanol in the presence of DO (e.g., at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%) and in the anoxic condition.

Some embodiments of the isolated bacteria include bacteria with a 16S rRNA gene that comprises the following partial sequence (“n” is a, c, g, or t):

(SEQ ID NO: 1)
taggtgtagg ggttgtcatg acctctgtgc cgccgctaac
gcattaagta ttccgcctgg ggagtacggt cgcaagatta
aaactcaaag gaattgacgg gggcccncac aagcagcgga
gcatgtggtt taattcgaag caacncgaag aaccttacct
agacttgaca tctcctgaat tacccttaat cggggaagcc
cttcggggna ggaagacagg tggtgcatgg ttgtcgtcag
ctcgtgtcgt gagatgttgg gttaagtccc gcaacgagcg
caacccttat tgttagttgc taccatttag ttgagcactc
tagcgagact gcccgggtta accgggagga aggtggggat
gacgtcaaat catcatgccc cttatgtcta gggctacaca
cgtgctacaa tggctggtac agagagatgn taaaccgtga
ggtggagcca aactttaaaa ccagtctcag ttcggattgt
aggctgaaac tcgcctacat gaagctggag ttgctagtaa
tcgcgaatca gaatgtcgcg gtgaatacgt tcccgggcct
tgtacacacc gcccgtcaca nnnnnnnnnt tggca

Some embodiments of the invention include compositions comprising the isolated bacteria. The compositions can comprise a percent relative to total bacteria that can be at least about 99.99%, at least about 99.90%, at least about 99.50%, at least about 99.00%, at least about 95.00%, at least about 90.00%, at least about 85.00%, at least about 75.00%, at least about 50.00%, at least about 25.00%, at least about 10.00%, or at least 1.00%. Some embodiments include compositions that are substantially pure. Substantially pure refers to the percent of the isolated bacteria relative to the total bacteria. Substantially pure can be at least about 99.99%, at least about 99.90%, at least about 99.50%, at least about 99.00%, at least about 95.00%, at least about 90.00%, at least about 85.00%, or at least about 75.00%.

Some embodiments of the invention include methods for producing one or more generated chemicals. In certain embodiments, the method includes the use of isolated bacterial strain TU-103. The growth media can be any suitable growth media, including but not limited to a synthetic broth, a semi-synthetic broth, or a nutrient broth. Some embodiments of the method for producing one or more generated chemicals include incubation at any suitable temperature, including but not limited to about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C. Some embodiments of the method for producing one or more generated chemicals include incubation with an initial concentration of energy-providing substrate at any suitable initial concentration, including but not limited to about 2 gl⁻¹, about 5 gl⁻¹, about 6 gl⁻¹, about 7 gl⁻¹, about 8 gl⁻¹, about 9 gl⁻¹, about 10 gl⁻¹, about 11 gl⁻¹, about 12 gl⁻¹, about 13 gl⁻¹, about 14 gl⁻¹, about 15 gl⁻¹, about 16 gl⁻¹, about 18 gl⁻¹, about 20 gl⁻¹, about 25 gl⁻¹, about 30 gl⁻¹, about 35 gl⁻¹, about 40 gl⁻¹, about 50 gl⁻¹, or about 60 gl⁻¹. The energy-providing substrate can be any suitable substrate including but not limited to carbohydrates, cellulose substrates, starch, glucose, or lactose.

Some embodiments of the method for producing one or more generated chemicals can use any suitable incubation method including but not limited to batch production, continuous production, use of a bioreactor, or a combination thereof. Some embodiments of the method include maintaining, regulating, or monitoring one or more suitable variables including but not limited to concentration of the energy-providing substrate, pH, DO concentration (e.g., keeping DO as close to 0% as possible, allowing DO to decrease (e.g., as it is consumed), or allowing DO to increase), biomass, temperature, energy-providing substrate consumed, or the concentration of one or more generated chemicals.

In some embodiments, the method is performed under aerobic conditions, under anaerobic conditions, or under a combination of both. In some instances, the method uses growth media with a percent dissolved oxygen (DO) of at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%. In some instances, the method uses growth media with an initial dissolved oxygen (DO) of at least about 0.1%, at least about 1.0%, at least about 5%, about 0.1%, about 0.4%, about 0.5%, about 1.0%, about 5%, about 6%, about 10%, about 18%, about 20%, about 21%, about 35%, about 50%, about 53%, about 60%, about 64%, about 65%, about 70%, about 80%, no more than 64%, no more than 70%, or no more than 85%. In some embodiments, the dissolved oxygen varies throughout the method.

In other embodiments of the method, the dissolved oxygen is removed from growth media by the isolated bacteria, which in some instances may result in anaerobic conditions. Dissolved oxygen can be removed by the isolated bacteria by any suitable mechanism, including but not limited to using superoxide dismutase (e.g., with any suitable superoxide dismutase activity including but not limited to about 0.05, about 0.10, about 0.12, about 0.15, about 0.20, about 0.30, or about 0.50 U per milligram protein) or using catalase. In some instances, the catalase activity is not dependent on the presence of hemin.

In some embodiments, the generated chemical can include but is not limited to butanol, acetone, butyric acid, ethanol, acetic acid, butanol, acetone, butyric acid, ethanol, acetic acid, ethanol, lactic acid, lactose, organic acids, alcohols, carbon dioxide, hydrogen, isopropanol, degradation products of cellulose or starch, or hydrolytic cleavage products of cellulose or starch. In some embodiments, one or more of the generated chemicals can be produced in any suitable amount, including but not limited to about 3.0 gl⁻¹, about 4.0 gl⁻¹, about 5.0 gl⁻¹, about 6.0 gl⁻¹, about 7.0 gl⁻¹, about 8.0 gl⁻¹, about 9.0 gl⁻¹, about 10.0 gl⁻¹, about 11.0 gl⁻¹, about 12.0 gl⁻¹, about 12.5 gl⁻¹, about 13.0 gl⁻¹, about 13.5 gl⁻¹, about 14.0 gl⁻¹, about 14.5 gl⁻¹, about 15.0 gl⁻¹, about 20.0 gl⁻¹, about 25.0 gl⁻¹, about 30.0 gl⁻¹, about 35.0 gl⁻¹, or about 40.0 gl⁻¹. In some embodiments, butanol can be produced in any suitable amount, including but not limited to at least about 3.0 gl⁻¹, at least about 5.0 gl⁻¹, at least about 10.0 gl⁻¹, about 3.0 gl⁻¹, about 4.0 gl⁻¹, about 5.0 gl⁻¹, about 6.0 gl⁻¹, about 7.0 gl⁻¹, about 8.0 gl⁻¹, about 9.0 gl⁻¹, about 10.0 gl⁻¹, about 11.0 gl⁻¹, about 12.0 gl⁻¹, about 12.5 gl⁻¹, about 13.0 gl⁻¹, about 13.5 gl⁻¹, about 14.0 gl⁻¹, about 14.5 gl⁻¹, about 15.0 gl⁻¹, about 20.0 gl⁻¹, about 25.0 gl⁻¹, about 30.0 gl⁻¹, about 35.0 gl⁻¹, or about 40.0 gl⁻¹. In some embodiments, acetone can be produced in any suitable amount, including but not limited to at least about 0.1 gl⁻¹, at least about 1.0 gl⁻¹, at least about 2.0 gl⁻¹, about 0.1 gl⁻¹, about 0.5 gl⁻¹, about 1.0 gl⁻¹, about 1.5 gl⁻¹, about 2.0 gl⁻¹, about 2.2 gl⁻¹, about 2.5 gl⁻¹, about 3.0 gl⁻¹, about 3.5 gl⁻¹, about 4.0 gl⁻¹, about 4.5 gl⁻¹, or about 5.0 gl⁻¹.

Other embodiments of the method include the use of one or more cellulose substrates, including but not limited to any suitable cellulose substrate, such as microcrystalline cellulose (C20), carboxymethyl cellulose (CMC), colloidal cellulose (CCOL), phosphoric acid treated cellulose (PASC), cotton fibers (CF), and filter paper (FP). Still other embodiments include the use of starch as a metabolite. Still other embodiments include methods that hydrolyze one or more cellulose substrate, metabolize starch, or both, and produce one or more generated chemicals (such as but not limited to butanol, acetone, or both).

Some embodiments of the invention include methods to isolate aerotolerant anaerobic bacteria from a sample. The sample may or may not comprise aerotolerant anaerobic bacteria. In some embodiments of this isolation method, the method comprises exposing a sample to an isolating atmosphere that comprises trace amounts of O₂ or any suitable amount of O₂ to prevent the isolation of strict anaerobes. In some embodiments, the amount of O₂ can be any suitable amount including but not limited to about 1 ppm O₂, about 2 ppm O₂, about 3 ppm O₂, about 4 ppm O₂, about 5 ppm O₂, about 6 ppm O₂, about 7 ppm O₂, about 8 ppm O₂, about 9 ppm O₂, about 10 ppm O₂, about 15 ppm O₂, about 20 ppm O₂, about 30 ppm O₂, about 50 ppm O₂, about 100 ppm O₂, about 150 ppm O₂, about 200 ppm O₂, or about 500 ppm O₂. In still other embodiments, the method of isolation includes the use of a glove box. In some instances, the method can regulate, maintain, or monitor the O₂ concentration in the isolating atmosphere. In still other instances, the O₂ concentration in the isolating atmosphere is not monitored. In some embodiments, the sample is exposed to heat (e.g., suitable heating the sample to kill all cells except endospores in the sample), ethanol (e.g., suitable exposure to ethanol to kill all cells except endospores in the sample), or both. In some embodiments, treatment with heat can comprise heating about 1 gram of sample at about 70° C. for about 15 minutes. In other embodiments, treatment with ethanol can comprise adding about 1 gram of sample to about 30 ml of about 50% (v/v) ethanol for about 1 hour.

### EXAMPLES

### Endospore and Bacteria Isolation

Approximately 10 g of animal feces or soil samples were collected in sterile plastic pouches and stored at −20° C. until further use. Feces of animals belonging to Equus burchellii (Zebra), Lama glama (Llama), Giraffidae giraffa (Giraffe) and Elephas maximus (Asian elephant) were obtained from the grounds of animal enclosures at the Audubon Zoo of New Orleans, USA. Environmental samples of wood mulch, pine mulch, ant mound and swamp mud were collected from a location (32790 Empire LA N lat 29° 23′ 32″, Long W 89° 36′ 11″) in Louisiana, USA. Further handling of fecal and environmental samples was done in an anaerobic glove box, under continuous flushing with 100% argon or dinitrogen atmosphere.

### Heat/Ethanol Treatment

All known butanol-forming clostridia produce heat and ethanol-resistant endospores. Thus, heat or ethanol treatments were designed to kill vegetative cells while leaving viable endospores that can germinate on agar plates and could be tested for butanol formation. For heat-treatment, 1 g of animal feces or soils were suspended in 30 ml sterile distilled water, mixed by vortexing for 1 minute, heated at 70° C. for 15 minutes and cooled to room temperature (Baer et al., Appl. Environ. Microbiol., Vol. 53, pp. 2854-2861 (1987)). Treatment with ethanol was done by adding approximately 1 g of the sample in a test tube containing 30 ml of 50% (v/v) ethanol (Nishida et al., Appl. Microbiol., Vol. 17, pp. 303-309 (1969)). The contents were mixed vigorously by vortexing and incubated at room temperature for 1 hour.

### Enrichment and Isolation of Bacterial Strains

One ml of heat-treated or 50% ethanol-treated samples was inoculated into 10 ml Reinforced Clostridial Medium (RCM; Difco) in 15 ml tubes with butyl rubber stoppers and screw caps. The tubes were flushed with argon gas and incubated at 35° C. for 5 days or until growth was observed, then the broth was diluted (10⁻³, 10⁻⁵) in sterile saline and plated on RCM plates with 2.5% (w/v) agar. These plates were incubated in anaerobic jars at 35° C. for 3-5 days or until isolated colonies were obtained. Cell morphology and gram character of the isolated colonies was determined by using the gram staining kit (Fluka) and the gram positive cells were stained by the Schaffer-Fulton spore staining technique for determining the spores (Motoya et al., J. Biotech., Vol. 79, pp. 117-126 (2000)). Gram positive cells were then re-inoculated into 10 ml T6 (Koransky et al., Appl. Environ. Microbiol., Vol. 35, p. 762 (1978)) broth with 6% (w/v) glucose (Kashket et al., Appl. Environ. Microbiol., Vol. 59, pp. 4198-4202 (1993)) in 15 ml glass tubes with an inverted Durham tube for gas detection. The tubes were flushed with argon gas, sealed with butyl rubber stoppers and screw caps and incubated with shaking at 35° C., 100 rpm for 5-7 days and 1 ml samples were removed at 24 hour intervals and analyzed for the presence of fermentation end products.

For short term storage, solventogenic bacterial isolates were maintained under refrigeration (4° C.) on CDC Anaerobe 5% sheep blood agar plates (BD Diagnostics, NJ, USA). For long-term storage, broth cultures were mixed with equal volumes of sterile 30% (v/v) glycerol and stored at −80° C. Inoculums used in fermentation studies were generated from single isolated colonies.

### Growth Media

Small-scale fermentations (175 ml) were carried out in T6 broth, P2 synthetic broth (Schaeffer et al., Science, Vol. 77, p. 194 (1933)) or P2Y semi-synthetic broth, all of which contain 60 gl⁻¹ of glucose. The composition of T6 media was Tryptone 6 gl⁻¹; yeast extract 2 gl⁻¹; KH₂PO₄ 0.5 gl⁻¹; MgSO₄.H₂O 0.3 gl⁻¹; FeSO₄.7H₂O 0.001 gl⁻¹; CH₃COONH₄ (38.9 mM) 3 gl⁻¹; cysteine HCl 0.5 gl⁻¹; pH 6.5. Components of P2 synthetic medium were prepared separately as Solutions 1, 2, 3 and 4. Solutions 1 (glucose 60 g; distilled water 790 ml) and 2 (K₂HPO₄ 0.5 g; KH₂PO₄ 0.5 g; CH₃COONH₄ 2.2 g and distilled water 100 ml) were sterilized separately and mixed. 10 ml of solution 3 (MgSO₄ 2 g; MnSO₄ 0.1 g; NaCl 0.1 g; FeSO₄ 0.1 g; distilled water 100 ml) and 1 ml of solution 4 (p-amino benzoic acid 0.01 g; thiamine 0.01 g; biotin 0.0001 g; distilled water 10 ml) were filter-sterilized and added to the mixture of sterile solutions 1 and 2. Semi-synthetic broth (P2Y) is P2 synthetic broth supplemented with 0.1 gl⁻¹ yeast extract. The pH of the medium (P2Y) prior to the initiation of fermentation was 6.5, and was not adjusted during fermentations.

### Conditions for Cell Growth and Chemical Production

To determine media compositions suitable for growth and for chemical production, T6, P2, and P2Y were made up to 165 ml in 175 ml Corning glass bottles (No. 1367) and inoculated with 10 ml of stationary phase TU-103 broth culture and incubated at 35° C., 100 rpm for 120 hours. Temperature optimization for growth and for acetone and butanol production was carried out by inoculating P2Y media, made up to 165 ml in 175 ml Corning glass bottles (No. 1367) with 10 ml of inoculum (grown in semisynthetic media) that was incubated at 26° C., 32° C., 35° C., 37° C., or 40° C. The dissolved O₂ tolerance for growth in P2Y media was determined by cultivating strain TU-103 in 175 ml Corning glass bottles (No 1367) with culture volumes of 20 ml, 100 ml, or 175 ml (and thus with air headspace volumes of 155 ml, 75 ml, 0 ml respectively) at 32° C., 100 rpm for 120 hours.

### Analytical Methods

The dry weight of cells was determined by centrifuging 1 ml of culture medium in a pre-weighed 1.5 ml eppendorf tube at 8000 rpm for 10 minutes. After removing the supernatant, the cell pellet was dried at 60° C. in an air convection oven until the weight failed to decrease (48-72 hours). All of the measurements were carried out in duplicate. The remaining culture supernatant was used for determining the glucose concentration by the dinitro salicylic acid (DNSA) method (Miller, Anal. Chem., Vol. 31, p. 426 (1959)). The DO concentration in broth was measured with a Mettler-Toledo polarographic O₂ probe, calibrated in the range of 0-100% by sparging with an inert gas (N₂) or air.

Chemicals produced in fermentations were quantified by Gas Chromatography-Mass Spectrometry. One ml of samples withdrawn from fermentations, supplemented with 1.13 μlml⁻¹ of 2-propanol (Malinkroft) as an internal standard, mixed, and centrifuged at 13,000 rpm, 4° C. for 10 minutes in a Sorvall SS34 rotor. The supernatant was filtered through a 0.2 μm (Acrodisc) syringe filter and analyzed to determine the chemical peak areas and the corresponding concentrations based on a standard curve. Chemicals in the fermentation samples were quantified using the method described by Thompson (Thompson, Enz. Microb. Technol., Vol. 13, p. 722 (1991)). Filtered supernatant samples were analyzed on a Varian CP-3800 gas chromatograph and a Varian (Saturn 2000) ion trap mass spectral detector with a Varian (CP 8410) auto injector. The GC conditions were set as described by Green et al. (Green et al., Microbiology, Vol. 142, pp. 2079-2089 (1996)) with some modifications. A ZB-wax capillary column (Phenomenex, Torrance, Calif.) of dimensions 30 m×0.25 mm×0.25 μm was used for the analysis under the following conditions: the initial column temperature of 35° C. was raised to 110° C. at 10° C./min, held for 3 min, further increased to 155° C. at 15° C. min⁻¹ and then to 200° C. at 20° C. min⁻¹. The temperature was held at 200° C. for 7.5 minutes. The injector temperature was set at 250° C. and helium was used as the carrier gas at a flow rate of 1 ml min⁻¹.

A standard curve was plotted by preparing aqueous mixtures of acetone, butanol and ethanol, each at concentrations of 1 μg ml⁻¹, 5 μg ml⁻¹, 10 μg ml⁻¹, 15 μg ml⁻¹ and 20 μg ml⁻¹ to which was added 1.13 μl ml⁻¹ of 2-propanol as an internal standard. The butanol and biomass yields (gg⁻¹) were calculated by dividing grams of butanol or biomass produced by the grams of glucose consumed.

Cell morphology was studied using a scanning electron microscope (SEM). Two to four μl of fermentation samples were placed on a metal SEM grid and allowed to dry in a laminar flow unit for 20-30 minutes. Grids were sputter coated with gold under vacuum and the samples were analyzed with a variable pressure Hitachi 3400 electron microscope and Hitachi 4800 high resolution SEM.

### DNA Extraction, PCR, and Nucleotide Sequence Analysis

After culturing strain TU-103 in T6 media at 32° C. for 24 hours, 1 ml of the cell suspension was centrifuged at 13,000-16,000 rpm for 2 minutes to obtain a cell pellet. Genomic DNA was purified from the cell pellet using the ArchivePure DNA Yeast & Gram-+Kit (5 PRIME Inc., Gaithersburg, Md., USA), using the method provided by the manufacturer. Oligonucleotide primer PCR A (5′-GGAGCAAACAGGATTAGATACC-3′ (SEQ ID NO:2); positions 774 to 795 [in the Escherichia coli rRNA numbering system]) and PCR B (5′-TGCCAACTCTATGGTGTGACG-3′ (SEQ ID NO:3) positions 1419 to 1403 [in the Escherichia coli rRNA numbering system]) were designed to amplify a relatively variable region of the 16S rRNA gene (Keis et al., Int. J. Syst. Bacteriol., Vol. 45, pp. 693-705 (1995)). The amplification was done using the protocol provided by Invitrogen Life Technologies (Carlsbad, Calif.). The contents of a 25 μl reaction mixture were: 10× AccuPrime™ PCR Buffer 12.5 μl, Primer Mix (10 μM each) 0.5 μl, template DNA 10 pg-200 ng, AccuPrime™ Taq DNA Polymerase 0.5 μl and autoclaved distilled water to make up the final volume to 25 μl. PCR's were performed on a Peltier thermal cycler (PTC 200). The initial conditions were set to 94° C. for 3 min; followed by 34 cycles of PCR amplification at 94° C. for 1 min, annealing at 55° C. for 1 min and extension at 72° C. for 1 min 30 sec. The samples were maintained at 72° C. for 8 min after which the temperature was lowered to 4° C. The PCR amplicons were purified using the ExoSAP-IT® purification kit (USB Corp. Cleveland, Ohio) using the manufacturer's instructions. A 10 μl aliquot of the PCR mixture was analyzed by electrophoresis in 1% (w/v) agarose gel containing 0.5 μg ml⁻¹ ethidium bromide to confirm the size of the amplified DNA product.

The nucleotide sequences of the primers A and B were chosen by the alignment of 16S rRNA gene sequences of C. acetobutylicum NCIMB 8052, C. beijerinckii DSM 79IT and NCIMB 9362T, C. botulinum (non-proteolytic) types B, E, and F, and C. butyricum ATCC 43755, DSM 2478, and NCIMB 8082 using the multiple sequence alignment program PILEUP of the Genetics Computer Group, Madison, Wis. (Keis et al., Int. J. Syst. Bacteriol., Vol. 45, pp. 693-705 (1995)).

The nucleotide sequence of the PCR products was determined at the DNA Sequencing Core of the Center for Gene Therapy, Tulane University Health Sciences Center, New Orleans, USA. The nucleotide sequence of the amplicon was compared against the sequences in EMBL sequence library using the Basic Local Alignment Search Tool (BLAST) (Altschul et al., J. Mol. Bio., Vol. 215, pp. 403-410 (1990)), in order to identify bacterial strains whose corresponding 16s rRNA gene sequence are most similar to that of strain TU-103

### Agar Plate Assay O₂ Sensitivity

100 ml semisynthetic broth media (P2Y) was inoculated with glycerol stocks of strain TU-103 or C. beijerinckii NCIMB 8052 cultures under an inert atmosphere and incubated at 32° C. for 48 hours under anaerobic conditions. One ml of the TU-103 culture from the exponential phase of growth was diluted 10⁻⁶-fold and 0.1 ml of the diluted culture was plated on anaerobic blood agar plates under anaerobic conditions. The plates containing cultures of strain TU-103 and C. beijerinckii NCIMB 8052 were exposed to air for 0, 15, 30, 45, and 60 minutes, incubated in an anaerobic jar at 32° C. for 24-48 hours and the bacterial colonies (cfu) were counted.

### Detection of Catalase and Superoxide Dismutase

For determining catalase activity, a loopful of colony material from colonies of strain TU-103 [grown on nutrient agar (Sigma) and anaerobic blood agar plates] and C. beijerinckii NCIMB 8052 [grown on anaerobic blood agar plates] was mixed with 100 μl of 3% hydrogen peroxide, on a glass slide and under an anaerobic atmosphere. The formation of gas bubbles was used as an indicator of catalase activity (Benson, Microbiological Applications, 8^{th} edition, McGraw Hill, New York (2002)). Production of O₂ gas during these assays was not quantified. For determining the intracellular SOD activity, bacteria were cultured in P2Y medium at 32° C., 100 rpm for 48 hours and cell free extracts were prepared using the protocol described by Schwarz et al. (Schwarz et al., J. Microbiol Methods, Vol. 68, pp. 396-402 (2007)). Cell free extracts were assayed for total protein content using the Bradford method (Bradford, Anal. Biochem., Vol. 72, pp. 248-254 (1976)), and for SOD activity using the EpiQuik™SOD Assay Kit (Epigentek, Brooklyn, N.Y.). SOD activity in the cell free extract is indicated by the SOD-mediated inhibition of the reaction between superoxide anion and formazon, which causes a brown discoloration of the reaction mixture that is quantified by measuring the absorbance at 470 nm. As the SOD concentration increases in this assay, the absorbance at 470 nm decreases. The SOD assays were done in 96 well microtitre plates and quantified using a Spectramax 150 plate reader.

### Results

A total of 14 solventogenic bacterial strains were isolated from various different samples of animal feces and soils. We isolated bacteria from the genus Clostridium, as all its species are able to produce heat and ethanol resistant endospores. Of the 14 solventogenic isolates, 10 were obtained using an ethanol-selective isolation procedure and the remaining 4 bacterial strains were obtained from samples that had been heat-treated. Cultivation of these 14 bacterial isolates under anaerobic conditions in T6 nutrient broth resulted in the formation of acetone and butanol. The strain that produced the highest levels of butanol was isolated from zebra feces and designated as TU-103 (ATCC ______).

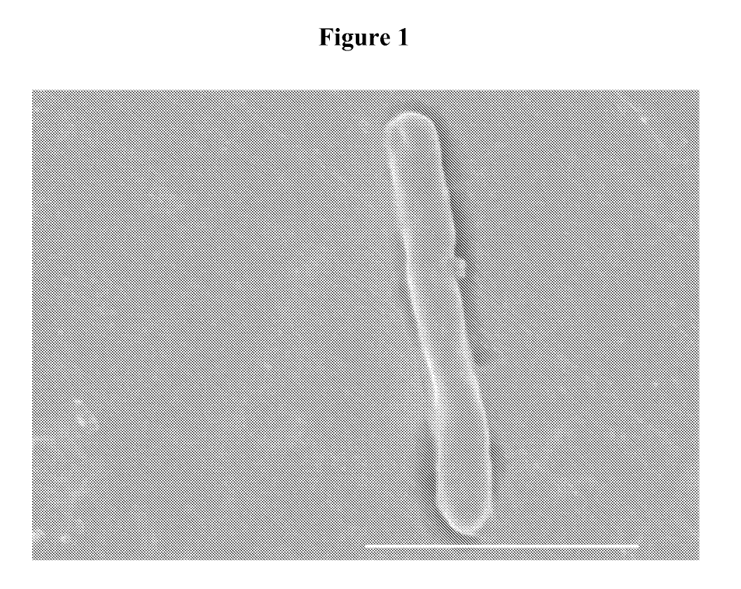

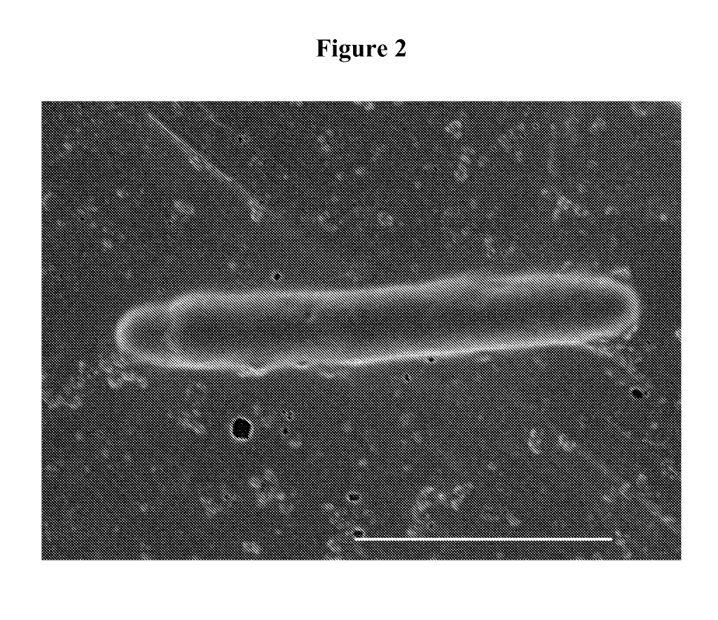

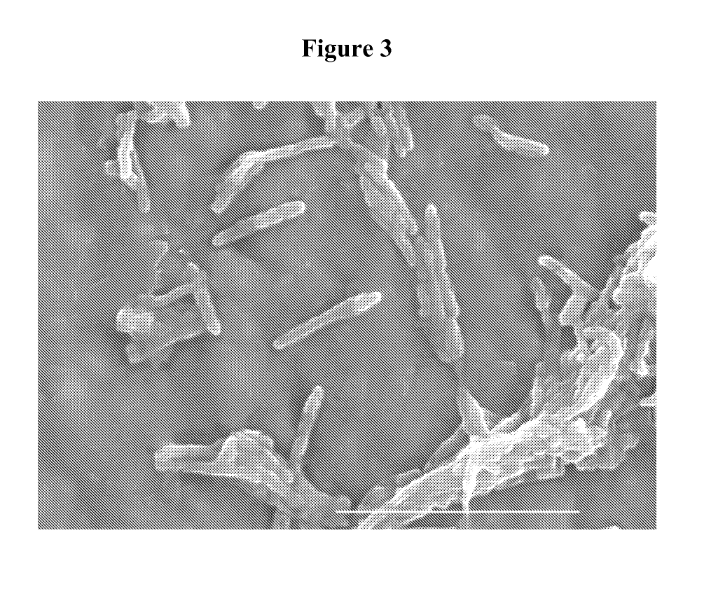

Cultivation of strain TU-103 under anaerobic conditions at 35° C. for 24 hours on blood agar plates resulted in the formation of pale gray β-hemolytic colonies with diameters of 5-10 mm that appeared to be dense at the center and had no outgrowths. Microscopic observations of material from colonies on RCM agar plates revealed the presence of motile gram positive rods, appearing as single cells or in short chains with or without apparent endospores. Cultivation of strain TU-103 in P2Y semisynthetic broth at 35° C. resulted in the appearance of rod-shaped cells with morphologies that depended on the age of the culture. Between 0 and 24 hr post-infection (PI), strain TU-103 formed vegetative rod-shaped cells (FIG. 1); whereas cigar-shaped stout rods were observed between 24 to 48 hours PI (post-innoculation) (FIG. 2), and sporulating rods were observed between 48 and 120 hours PI (FIG. 3).

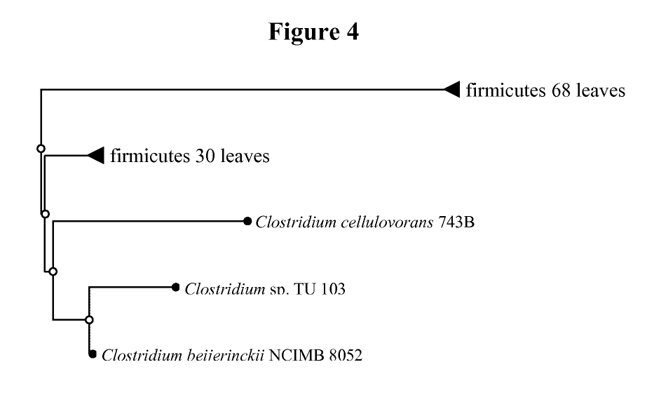

BLAST analysis (Altschul et al., J. Mol. Biol., Vol. 215, pp. 403-410 (1990)) of the nucleotide sequence of a phylogenetically informative segment of the 16S rRNA of strain TU-103 revealed that it appears most similar to that of Clostridium beijerinckii NCIMB 8052 (99%) and Clostridium cellulovorans (94%) (FIG. 4). The results of the nucleotide sequence comparisons in combination with the morphological properties and ability to produce butanol suggests that strain TU-103 is a bacterium that belongs to the Genus Clostridium. The 16S rRNA sequence of strain TU-103, described in this work has been deposited in GenBank under accession number HM447592.

As a step toward identifying the conditions that optimize growth, acetone production, and butanol production by strain TU-103, the role of broth medium composition and temperature were investigated. Strain TU-103 was cultivated in nutrient broth (T6), synthetic broth (P2) and semi-synthetic broth (P2Y) at 35° C. for 120 hours. In T6 and P2Y broths, strain TU-103 produced higher cell yields, acetone production, and butanol production than in P2 broth cultures (Table 1a). Among the three growth media tested, the biomass yields, acetone production, and butanol production were similar in T6 and P2Y broth but were lower in P2 broth (Table 1a).

TABLE 1a
Cell growth and acetone and butanol formation during anaerobic
cultivations of TU-103 isolate at 35° C. for 120 hours
in different media.

| | Concentrations (gl⁻¹) | | |
|---|---|---|---|
| Cultivation media | Biomass | Butanol | Acetone |
| T6 | 1.8 ± 0.2 | 12.0 ± 0.2 | 3.4 ± 0.3 |
| P2 | 0.9 ± 0.1 | 0.4 ± 0.3 | 0.1 ± 0.1 |
| P2Y | 1.7 ± 0.3 | 10.8 ± 0.2 | 3.7 ± 0.2 |

Yields of biomass, acetone production, and butanol production obtained during cultivations of strain TU-103 in P2Y broth at temperatures between 32° C. and 37° C. are comparable and significantly higher than in cultures incubated at 26° C. (Table 1b).

TABLE 1b
Effect of temperature on cell growth and acetone and butanol
production during anaerobic cultivations of strain TU-103 in
semisynthetic medium (P2Y broth) after 120 hr.

| | Concentrations (gl⁻¹) | | |
|---|---|---|---|
| Temperature (° C.) | Biomass (dry wt.) | Butanol | Acetone |
| 26 | 0.9 ± 0.1 | 3.0 ± 0.7 | 0.5 ± 0.3 |
| 32 | 1.6 ± 0.3 | 13.5 ± 0.3 | 2.2 ± 0.2 |
| 35 | 1.5 ± 0.1 | 13.0 ± 0.1 | 1.0 ± 0.7 |
| 37 | 1.5 ± 0.2 | 13.0 ± 0.4 | 2.0 ± 0.3 |
| 40 | 1.2 ± 0.3 | 5.0 ± 0.7 | 1.0 ± 0.1 |

While investigating the culture conditions of strain TU-103, we discovered that it was able to form healthy-looking colonies on agar plates that were incubated under aerobic conditions. For comparing the effects of exposure to O₂ on strain viability, anaerobic blood agar plates seeded with cells of strain TU-103 or C. beijerinckii NCIMB 8052 were exposed to air for different time intervals, and then transferred to an anaerobic incubator where colonies were allowed to form. The results of these exposures indicate that the colony forming units (cfu) of strain TU-103 were not affected by exposure to air for 15, 30, 45 or 60 minutes because they were identical in number to those that formed on agar plates that were not exposed to air. On the other hand, the cfu of C. beijerinckii NCIMB 8052 decreased by approximately 5-fold from 5×10⁶ cfu to 1×10⁶ cfu after 15 minutes of air-exposure, and no colonies formed after exposure to air for 30 min or longer (Data not shown).

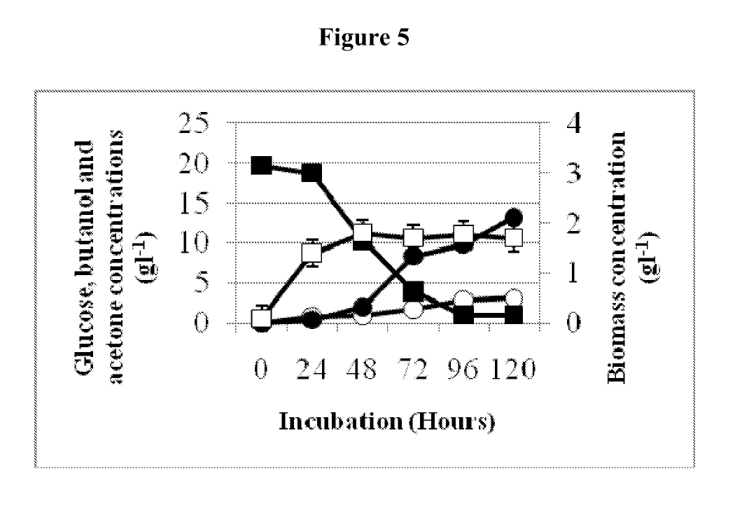

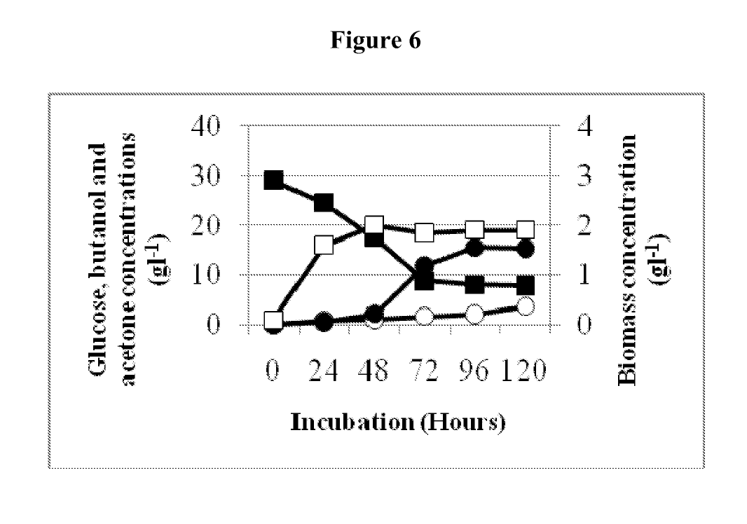

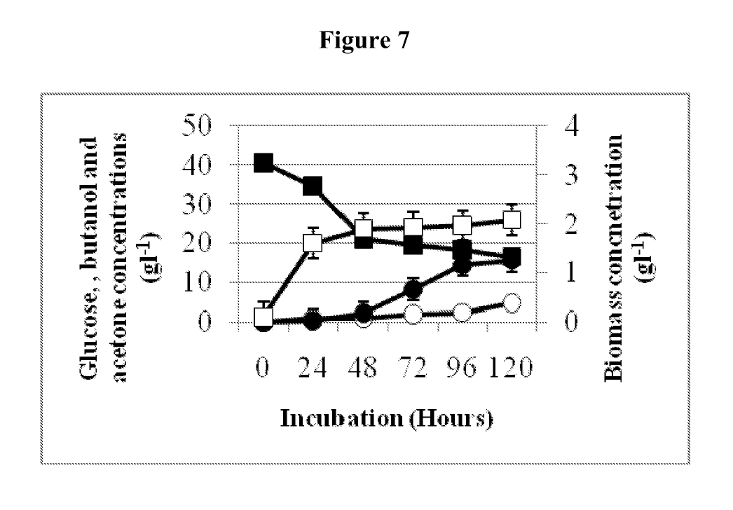

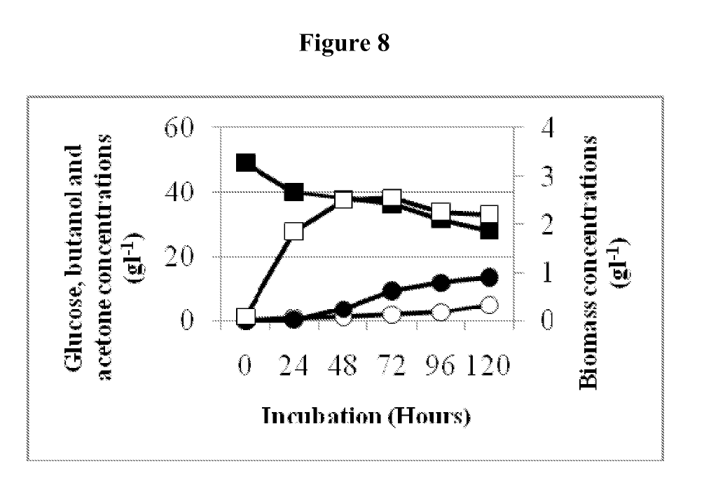

The effects of glucose concentration on cell growth, butanol production, and acetone production was determined by cultivating strain TU-103 for 120 hr at 32° C. under anaerobic conditions in P2Y broth containing 20, 30, 40, or 50 gl⁻¹ of glucose (FIG. 5, FIG. 6, FIG. 7 and FIG. 8, respectively). Of the four conditions tested, the highest concentrations of glucose metabolized (23.5 gl⁻¹) and acetone plus butanol produced (20.4±0.5 gl⁻¹) was in broth containing 40 gl⁻¹ glucose (FIG. 7).

As the concentration of glucose increased from 20 to 50 gl⁻¹ in P2Y broth there was accompanying steady decline in the percentage of glucose metabolized ranging from 95% at 20 gl¹ to 44% at 50 gl⁻¹, whereas the ratio of the concentration of acetone plus butanol produced per amount of glucose metabolized was approximately 0.86 in each case. Although acetone and butanol yields were similar in broth cultures supplemented with glucose, the lowest yield (16.3 gl⁻¹) came from cultures with 20 gl⁻¹ of glucose. Cultures growing in broth with 40 gl⁻¹ of glucose metabolized the most glucose (23.5 gl⁻¹) and not surprisingly produced the highest yield of acetone plus butanol (20.4±0.5 gl⁻¹). The highest yield of acetone plus butanol per gram of cell mass was achieved in broths with 30 gl⁻¹ of glucose.

The yields of cell mass during cultivations of strain TU-103 in 20, 30, and 40 gl⁻¹ of glucose were similar, and maximum yields were attained in the initial 48 hours of cultivation after which the bacterial culture remained in the stationary phase of growth (FIG. 5, FIG. 6, and FIG. 7). The cell growth pattern in P2Y broth with 50 gl⁻¹ of glucose was somewhat different from that observed at lower glucose concentrations because the maximum cell yield of 2.5±0.2 gl¹ was obtained at 72 hr PI which was followed by a decrease in cell concentrations to 2.2±0.1 gl⁻¹ over the next 24 hours. Thereafter, the cell concentration remained unchanged (FIG. 8).

The effects of dissolved oxygen on growth and on acetone and butanol production were tested. Aerobically grown TU-103 colonies were used to build small inoculums that were then used for anaerobic cultivations. Analysis of such cultures revealed that there was no reduction in cell growth, acetone production, or butanol production caused by exposure of cultures to O₂ (data not shown).

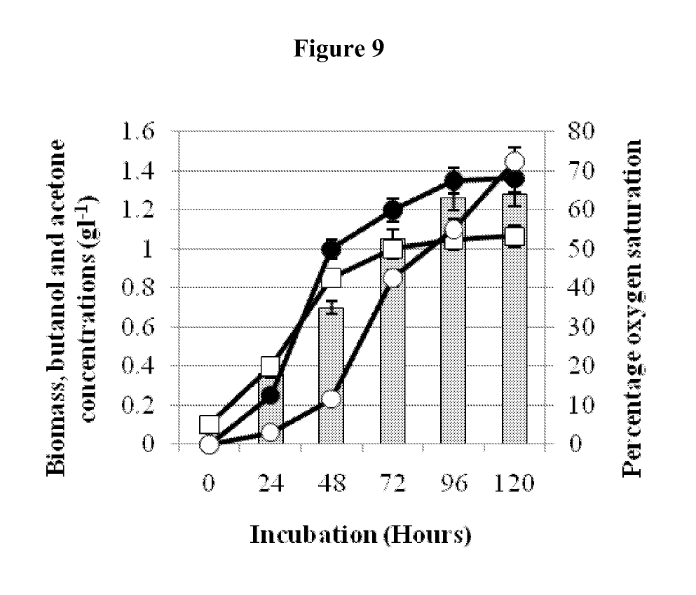

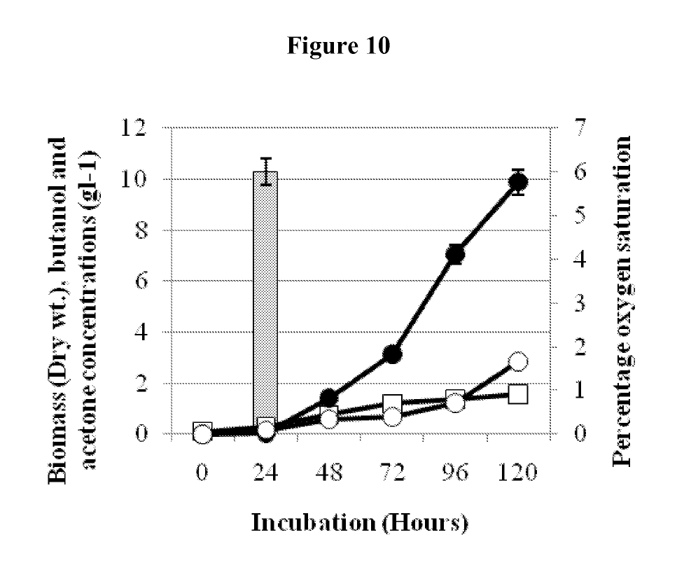

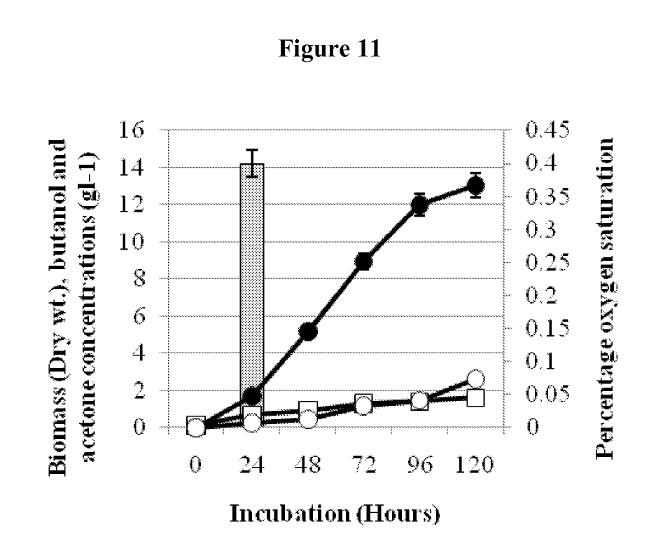

To investigate the effects of dissolved O₂ (DO) on growth and on acetone and butanol production, strain TU-103 was cultured in P2Y broth with air headspace volumes of 89%, 43%, and 0% (FIG. 9, FIG. 10, and FIG. 11, respectively). The DO concentration measurements in cultures (with 89% air headspace) showed that the DO concentrations increased steadily over time and reached 64% saturation by 120 hr PI (FIG. 9). Similar DO estimations in 100 ml cultures with approximately 43% air headspace showed that the broth reached approximately 6% saturation with O₂ by the 24 hr PI after which DO was not detected (FIG. 10). Cultivations of TU-103 in broth with no air headspace did not result in complete anaerobiosis because 0.4% DO was detected in the broth 24 hours after inoculation, however, no dissolved O₂ was detected thereafter (FIG. 11).

The biomass (0.67±0.3 gl⁻¹) and acetone plus butanol concentrations (1.7±0.1 gl⁻¹) obtained at 24 hr PI in cultures with no air headspace, despite the presence of 0.4% DO, increased to maximum levels of 1.6±0.1 gl⁻¹ and 15±0.2 gl⁻¹ respectively (FIG. 11) by 120 hr PI. Increased DO of up to 6% during the initial 24 hours of cultivation in broth with approximately 43% air headspace reduced yields of biomass (0.3±0.3 gl⁻¹) and acetone plus butanol (0.7±0.05 gl⁻¹) (FIG. 10), but continued incubation of the cultures until 120 hr PI was accompanied by the elimination of DO in the broth resulting in an increase in cell mass and acetone plus butanol concentrations to 1.5 gl⁻¹ and 11.8±0.5 gl⁻¹ respectively (FIG. 10). As expected, the concentrations of DO in the culture medium increased over time upon increasing the air headspace volume to 89% (FIG. 9). Despite the increase in the dissolved O₂ concentrations from 18% at 24 hr PI to 64% at 120 hr PI, strain TU-103 initiated growth, acetone formation, and butanol formation and resulted in cell yields and acetone plus butanol concentrations of 1.06±0.1 gl⁻¹ and 2.8±0.2 gl⁻¹ respectively (FIG. 9).

### Catalase and SuperOxide Dismutase(SOD) Studies

SOD and catalase can play roles in protecting cells from the deleterious effects of activated species of O₂ such as superoxide anion and hydrogen peroxide. We therefore measured and compared the SOD activities in strain TU-103 and C. beijerinckii NCIMB 8052. The SOD activities of strain TU-103 and C. beijerinckii were 0.12±0.06 U and 0.03±0.02 U per milligram of protein, respectively. Strain TU-103 showed a positive catalase reaction when 3% hydrogen peroxide was dripped over colony material taken from colonies grown on nutrient or anaerobic blood agar plates, as indicated by the rapid and vigorous formation of gas bubbles. In contrast, no evidence of catalase activity was observed after addition of 3% hydrogen peroxide onto colonies of C. beijerinckii NCIMB 8052.

### Bioreactor Studies

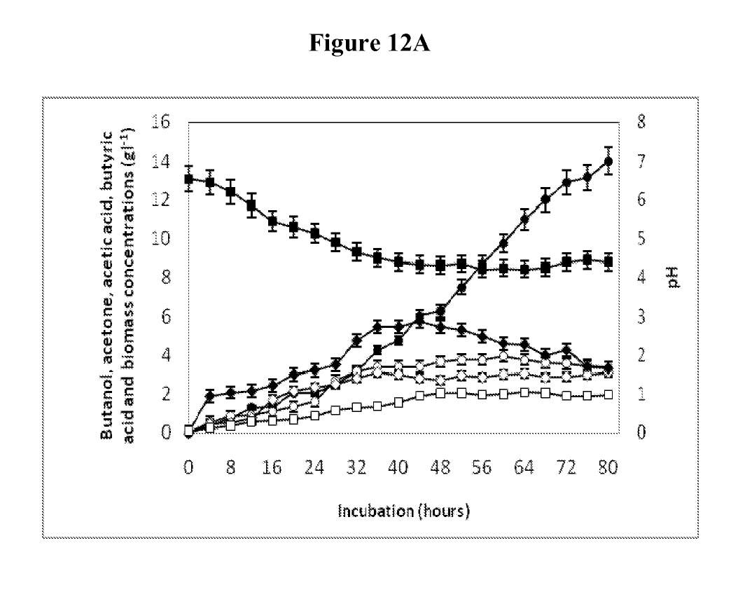

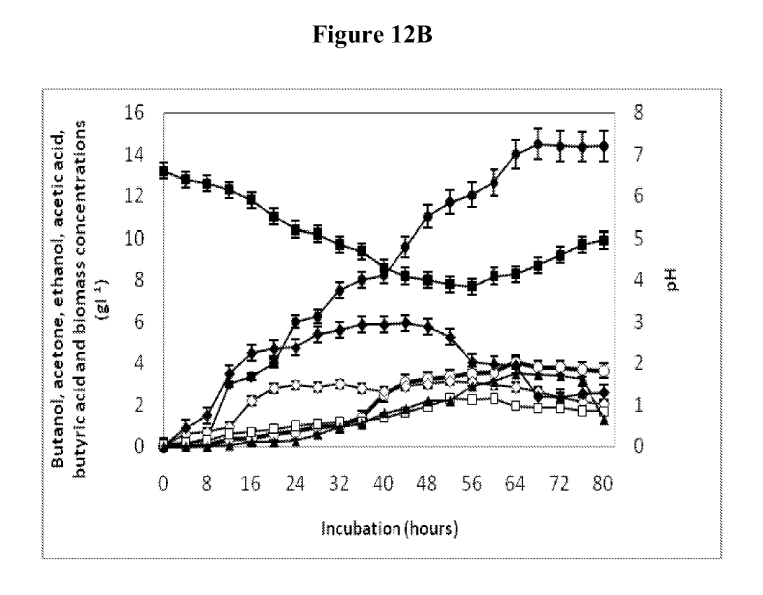

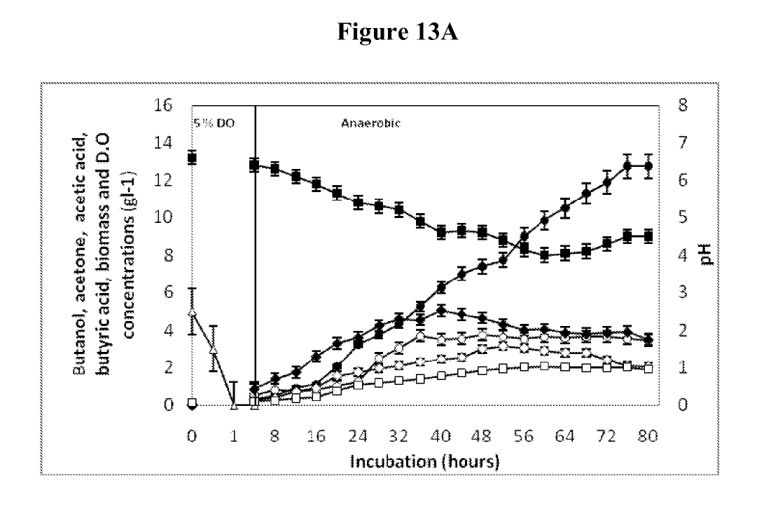

After a 10 l stirred tank bioreactor under anaerobic conditions was inoculated with strain TU-103, the pH of the P2Y broth dropped gradually from 6.5 to 4.3 over the first 60 hours PI (FIG. 12A). The levels of butyric acid reached 5.7±0.2 gl⁻¹ by 48 hours PI and then decreased to 3.2±0.3 gl⁻¹ by 80 hours of fermentation. In contrast, the acetic acid levels increased to 2.9±0.1 gl⁻¹ by 36 hours and remained relatively constant until the fermentation was terminated (FIG. 12A). Under anaerobic conditions, the cell mass of strain TU-103 increased steadily over the first 48 hours of fermentation, reached a concentration of 2.05±0.05 gl⁻¹ and remained unchanged thereafter (FIG. 12A). Low amounts (0.1±0.01 gl¹) of butanol and acetone were detected at the beginning of anaerobic cultivations, probably represented carry-over from the inoculum. Butanol levels peaked at 14 gl⁻¹ by 76 hr of fermentation (FIG. 12A). The maximum concentration of acetone obtained during anaerobic fermentations of strain TU-103 was 3.95±0.2 gl⁻¹. The total glucose consumed during anaerobic fermentations of strain TU-103 was 58.8 gl⁻¹. The butanol and biomass yields obtained during anaerobic cultivations of strain TU-103 were 0.23 gg⁻¹ and 0.034 gg⁻¹ respectively, while the butanol productivity was 0.18 gl⁻¹h⁻¹ (Table 2). Ethanol was not detected as a fermentation end-product of strain TU-103.

TABLE 2
Fermentation parameters of C. beijerinckii NCIMB 8052
and Strain TU-103 under different culture conditions.

| | | Maximum | Glucose | Butanol | Biomass | Butanol |
|---|---|---|---|---|---|---|
| | Fermentation | butanol conc. | consumed | yields* | yields* | productivity |
| Bacteria | conditions | (gl−1) | (gl−1) | (gg−1) | (gg−1) | (gl−1h−1) |
| TU-103 | Anaerobic | 14 ± 0.05 | 58.8 ± 0.05 | 0.23 ± 0.02 | 0.034 ± 0.003 | 0.21 ± 0.07 |
| | 5% DO | 12.7 ± 0.03 | 58.7 ± 0.2 | 0.21 ± 0.005 | 0.034 ± 0.002 | 0.18 ± 0.02 |
| | 21% DO | 7.05 ± 0.03 | 57.5 ± 0.2 | 0.12 ± 0.01 | 0.020 ± 0.002 | 0.16 ± 0.03 |
| C. beijerinckii | Anaerobic | 14.5 ± 0.3 | 58 ± 0.3 | 0.25 ± 0.02 | 0.039 ± 0.001 | 0.09 ± 0.01 |
| NCIMB 8052 | | | | | | |

*Yields expressed as grams of butanol/biomass produced per gram of glucose consumed.

Growth of C. beijerinckii NCIMB 8052 in the 10 l bioreactor under anaerobic conditions in P2Y broth was accompanied by a gradual drop in the pH from 6.5 to 3.8 by 56 hours PI (FIG. 12B). The maximum concentrations of butyric (approximately 44 hr PI) and acetic acid (approximately 36 hr PI) produced by C. beijerinckii were both comparable to the levels obtained during anaerobic fermentations of strain TU-103 (FIG. 12). The cell mass (2.3 gl^{-1±0.07} gl⁻¹) produced during anaerobic cultivations of C. beijerinckii was also comparable to the cell concentrations obtained during anaerobic fermentations of strain TU-103 (FIG. 12). In anaerobic cultures of C. beijerinckii, small amounts of acetone, butanol and ethanol were detected at the onset of cultivations, and maximum butanol levels of 14.5±0.2 gl⁻¹ were obtained by 60 hours PI (FIG. 12B). The highest concentrations of acetone (4.05±0.1 gl⁻¹) in anaerobically grown bioreactor cultures of C. beijerinckii were similar to the levels obtained in TU-103 cultures, cultivated under the same conditions. The formation of ethanol (1.85±0.08 gl⁻¹) was detected in C. beijerinckii cultures. The levels of glucose consumed during anaerobic fermentations of C. beijerinckii were 58 gl⁻¹ and the butanol and biomass yields were 0.25 gg⁻¹ and 0.039 gg⁻¹ respectively, whereas the fermentation productivity was 0.09 gl⁻¹h⁻¹ (Table 2).

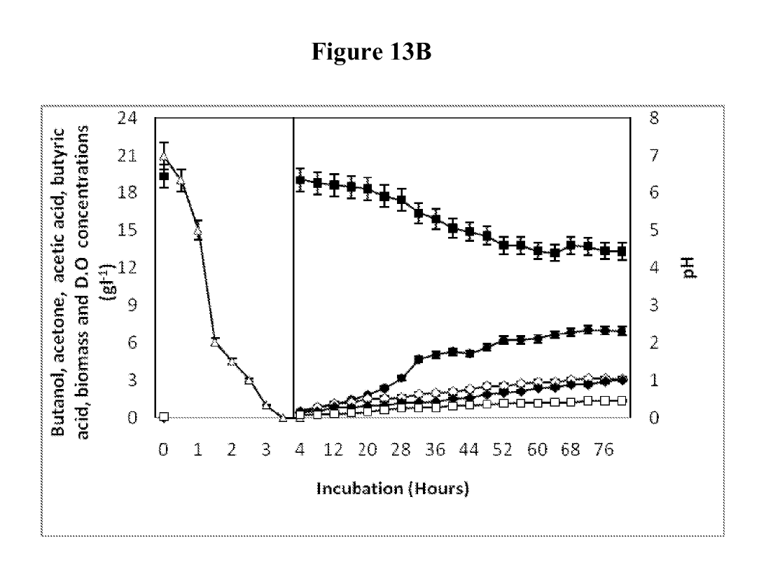

The effects of dissolved oxygen on cell growth and chemical formation was investigated by inoculating strain TU-103 into the 10 l bioreactor containing P2Y broth with DO levels adjusted to 5% or 21% (FIG. 13) but not adjusted thereafter. On inoculation of strain TU-103 in broths containing 5% DO levels, rapid de-oxygenation of the broth occurred causing the DO levels to drop to 2% in the initial 30 minutes, and to 0% within the first hour of fermentation (FIG. 13A). Thereafter, the N₂ supply was restored and the growth of strain TU-103 resumed and cell growth was accompanied with a drop in pH from 6.5 to 4.0 by 60 hours PI (FIG. 13A). The combined levels (8.2±0.7 gl⁻¹) of peak acetic acid (approximately 40 hours PI) and butyric acid (approximately 52 hr PI) produced during strain TU-103 cultivations in broth with 5% initial DO (FIG. 13A) was comparable to the acid levels (8.6±0.3 gl⁻¹) obtained during anaerobic fermentations of TU-103 (FIG. 12A). Strain TU-103 produced lower butanol concentrations (12.75±0.03 gl⁻¹) (FIG. 13A) under 5% initial DO levels than under anaerobic cultivations. The acetone (3.7±0.2 gl⁻¹) and biomass (2.0±0.1 gl⁻¹) levels produced by strain TU-103 under 5% initial DO levels were however comparable to their levels obtained during anaerobic cultivations (FIG. 12A). On cultivating strain TU-103 under 5% initial DO levels, 58.7 gl⁻¹ of glucose was utilized and the corresponding butanol yield and the fermentation productivity was reduced by 8% (0.21 gg⁻¹) and 24% (0.16 gl⁻¹h⁻¹) as compared to cultivations under complete anaerobiosis (Table 2), whereas the biomass yields under both conditions were similar (0.034 gg⁻¹) (Table 2). Fermentation runs were also conducted to determine the effect of sparging the bioreactor broth with N₂ after TU-103 cultures had established anaerobic conditions by scavenging the O₂ from broth with an initial DO of 5%. When the broths were not sparged with N₂, results showed a decrease in acetic and butyric acid levels and the butanol (0.18 gg¹) and biomass (0.030 gg⁻¹) yields were reduced (Data not shown).

To further investigate the aerotolerance of strain TU-103, we analyzed cell growth and chemical formation in a 10 l bioreactor with broth at a starting DO content of 21%. As seen during fermentations with initial DO concentrations of 5%, rapid de-oxygenation of the broth was observed. On inoculation of strain TU-103, in broth with an initial DO level of 21%, the DO in the cultivation medium decreased by 79% in the first 2 hours, and no DO was detected by 3.5 hours PI (FIG. 13B). Cultivations of strain TU-103 at 21% initial DO levels caused the pH to drop from 6.5 to 4.4 by 64 hours PI. The total peak acetic and butyric acid levels (6.0±0.5 gl⁻¹) obtained in strain TU-103 fermentations conducted at initial levels of 21% DO were lower than the total peak acid levels (8.2±0.7 gl⁻¹) obtained during cultivations under 5% DO (FIG. 13). As compared with TU-103 fermentations under initial DO levels of 5%, cultivations under 21% initial DO levels caused a decrease in the levels of biomass (1.35±0.05 gl⁻¹) and butanol (7.05±0.03 gl⁻¹), while the acetone levels were higher (4.8±0.3 gl⁻¹). Resultantly, the yields of butanol (0.12 gg⁻¹) and biomass (0.02 gg⁻¹) and the fermentation productivity were reduced on cultivating strain TU-103 under initial DO levels of 21% than under 5% (Table 2). As observed with TU-103 cultures in broth with initial DO levels of 5%, N₂ sparging was also found to improve the yield of biomass and chemicals during the anaerobic phase of growth in broth with initial DO levels of 21%. Failure to sparge the fermentation broth during the anaerobic phase of the fermentation caused a decrease in acetic and butyric acid levels and reduced the yields of butanol (0.05 gg⁻¹) and biomass (0.01) (data not shown).

In contrast to strain TU-103 which grew and produced acetone and butanol in broths saturated with initial DO levels of 5% or 21%, cells of C. beijerinckii were unable to grow in presence of 5% DO and no acid or chemical formation was detected.

### Cellulose Substrate Hydrolysis

We investigated the ability of cultures of strain TU-103 to hydrolyze a variety of different cellulose substrates including: microcrystalline cellulose (C20), carboxymethyl cellulose (CMC), colloidal cellulose (CCOL), phosphoric acid treated cellulose (PASC), cotton fibers (CF), and filter paper (FP). These cellulose substrates were tested to determine the cellulolytic activity of TU-103. Five percent (v/v) of TU-103 culture was added as the inoculum to a Corning glass culture bottle containing sterile P2Y medium (total volume 175 ml) with approximately 1 g of cellulosic substrate, the culture was briefly bubbled with nitrogen, sealed and incubated on a shaker incubator at 32° C., 100 rpm for a period of 248 hours. Three ml samples were withdrawn from the culture bottles at 0, 24, 48, 72, 96, 120, 148, 172, 196, 224, and 248 hours. One ml of the sample was used to determine the dry weight of cells. The second 1 ml sample was centrifuged, filtered, and 1.13 μl of isopropanol was added as an internal standard and the sample analyzed by gas chromatography to detect butanol and other chemicals (e.g., organic acids). The third 1 ml sample was used to determine the concentration of reducing sugars in the culture medium (using the DNSA method) and cellulase activities were measured using a Filter paper assay. GC analysis of the culture samples showed in the metabolite profile displayed in Table 3.

TABLE 3
Metabolite profiles show chemicals produced
from each cullulosic substrate

| | Cellulosic substrates | | | | | |
|---|---|---|---|---|---|---|
| Metabolites | C20 | CMC | CCOL | PASC | FP | CF |
| Ethanol | X | X | X | ◯ | ◯ | ◯ |
| Acetone | ◯ | ◯ | X | ◯ | ◯ | X |
| Butanol | ◯ | ◯ | ◯ | ◯ | ◯ | X |
| Acetic acid | ◯ | X | X | X | X | X |
| Butyric acid | ◯ | X | X | X | X | X |

X indicates chemical is produced;
◯ indicates chemical is not produced

During cultivation of TU-103 in P2Y broth containing C20, only ethanol was detected after 48 hours of incubation, whereas organic acids such as acetic and butyric acids were detected along with ethanol in P2Y cultures containing CMC. TU-103 cultures containing CCOL as the substrate resulted in the formation of ethanol, acetone, acetic acid, and butyric acid after 48 hours of incubation. Incubation of TU-103 in presence of PASC and FP resulted in the formation of acetic acid and butyric acid whereas neither acetone nor butanol was produced. On cultivation of TU-103 in presence of CF, the production of acetic and butyric acid was accompanied with the formation of acetone (0.03 gl⁻¹) and butanol (0.17 gl⁻¹) in the medium. Analysis of the culture samples for reducing sugar concentrations by the DNSA method indicated that low concentrations of reducing sugars could be detected; these sugars are produced by hydrolysis of cellulose and their level is relatively low throughout the fermentation, which might be due to rapid consumption of the released sugars by the organism.

During fermentation, cellulase activity is detected in cultures from 20 hours until 120 hours post-inoculation and an approximate enzyme activity of 0.03 U/ml was obtained in all of the cultures.

### Metabolism of Starch

We found that strain TU-103 has the ability to utilize starch as a source of carbon for growth, and it has the ability to form colonies on starch containing agar plates incubated at 32° C. in an anaerobic chamber.

Using the following procedure, TU-103 showed an ability to metabolize starch on agar plates. Agar plates with P2Y broth medium containing 1% starch were prepared. TU-103 culture was then streaked onto the starch containing agar plates. The agar plates were incubated at 32° C. for 24-48 hours or until bacterial colonies were visible. Starch degradation was detected by addition of an iodine solution to the agar plates in the region where the colonies were growing. In this assay, regions on the agar plates where starch was actively degraded were revealed by a lack of staining by iodine. These agar plates displayed large unstained-by-iodine zones around the colonies growing on the agar plates, thus indicating that TU-103 actively hydrolyzed starch.

Liquid cultures of strain TU-103 were set up in 175 ml glass bottles containing 16 ml of P2Y broth supplemented with 1% starch. The broth was sparged with N₂ gas for 5 minutes to establish anaerobic conditions, inoculated with 10 ml of TU-103, and incubated at 32° C., 100 rpm. The culture broth was analyzed for the presence of butanol, acetone, acetic acid, and butyric acid. Starch containing broth cultures of strain TU-103 produced acetone, butanol, acetic acid, and butyric acid as end-products of fermentation after incubation at 32° C., 100 rpm for 180 hours.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the specification, “a” or “an” may mean one or more. As used in the claims, when used in conjunction with the word “comprising,” the words “a” or “an” may mean one or more than one. As used herein “another” may mean at least a second or more. As used in the specification, the phrases “such as” and “e.g.” mean “for example, but not limited to” in that the list following “such as” or “e.g.” provides some examples but is not necessarily a fully inclusive list.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as a basis for the claims and as a representative basis for teaching one skilled in the art to employ the present invention in any appropriate manner.

What is claimed is:

1. Isolated bacteria of the Clostridium genus, where the isolated bacteria (1) are aerotolerant and (2) can produce one or more generated chemicals; wherein one of the one or more generated chemicals is butanol.

2. The isolated bacteria of claim 1, wherein the isolated bacteria are TU-103 (ATCC ______).

3. The isolated bacteria of claim 1, wherein the 16S rRNA gene of the isolated bacteria comprises SEQ ID NO:1.

4. The isolated bacteria of claim 1, wherein one or more generated chemicals further comprises one or more selected from the group consisting of acetone, butyric acid, and acetic acid.

5. The isolated bacteria of claim 1, wherein the isolated bacteria can produce butanol at a concentration of at least about 3.0 gl⁻¹.

6. The isolated bacteria of claim 1, wherein the isolated bacteria can produce acetone at a concentration of at least about 0.1 gl⁻¹.

7. The isolated bacteria of claim 1, wherein the isolated bacteria can survive in a growth media comprising a percent of dissolved oxygen (DO) that is at least 0.1%.

8. The isolated bacteria of claim 1, wherein the isolated bacteria can grow in a growth media comprising a percent of DO that is at least 0.1%.

9. The isolated bacteria of claim 1, wherein the isolated bacteria can survive exposure to air for at least about 1 minute.

10. The isolated bacteria of claim 1, wherein the isolated bacteria can hydrolyze one or more cellulose substrates.

11. The isolated bacteria of claim 1, wherein the isolated bacteria can metabolize starch.

12. A composition comprising the isolated bacteria of claim 1.

13. The composition of claim 11, wherein the isolated bacteria is substantially pure.

14. A method for producing one or more generated chemicals comprising
growing the isolated bacteria of claim 1 in a growth media, and
recovering at least one of the one or more generated chemicals.

15. The method of claim 14, wherein the one or more generated chemicals comprises one or more selected from the group consisting of butanol, acetone, butyric acid, and acetic acid.

16. The method of claim 14, wherein DO in the growth media is at least 0.1%.

17. The method of claim 14, wherein dissolved oxygen is removed from the growth media by the isolated bacteria.

18. The method of claim 14, wherein growing the isolated bacteria is under anaerobic growth conditions.

19. The method of claim 14, wherein butanol is produced in amount of at least 3.0 gl⁻¹.

20. The method of claim 14, wherein acetone is produced in an amount of at least 0.1 gl⁻¹.

21. The method of claim 14, wherein the isolated bacteria hydrolyzes one or more cellulose substrates.

22. The method of claim 14, wherein the isolated bacteria metabolizes starch.

23. A method for isolating aerotolerant bacteria from a sample comprising exposing a sample to an isolating atmosphere that comprises at least trace amounts of O₂.

24. The method of claim 23, wherein the isolating atmosphere is in a glove box.

25. The method of claim 23, wherein the method further comprises exposing the sample to heat, ethanol, or both.


SEQUENCE LISTING (3 sequences)

SEQ ID NO: 1 - DNA, 595 bp, Closterium
  misc_feature (107)..(107): n is a, c, g, or t
taggtgtaggggttgtcatgacctctgtgccgccgctaacgcattaagtattccgcctggggagtacggtcgcaagattaaaactcaaaggaattgacgggggcccncacaagcagcggagcatgtggtttaattcgaagcaacncgaagaaccttacctagacttgacatctcctgaattacccttaatcggggaagcccttcggggnaggaagacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggctggtacagagagatgntaaaccgtgaggtggagccaaactttaaaaccagtctcagttcggattgtaggctgaaactcgcctacatgaagctggagttgctagtaatcgcgaatcagaatgtcgcggtgaatacgttcccgggccttgtacacaccgcccgtcacannnnnnnnnttggca

SEQ ID NO: 2 - DNA, 22 bp, Artificial Sequence
  Primer for PCR
ggagcaaacaggattagatacc

SEQ ID NO: 3 - DNA, 21 bp, Artificial Sequence
  Primer for PCR
tgccaactctatggtgtgacg